(19) United States
(12) Patent Application Publication
(10) Pub. No.: US 2002/0055733 A1
(43) Pub. Date: May 9, 2002

(54) Catheter assembly and method of use

(76) Inventor: W. Stan Wilson, Missoula, MT (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/022,018

(22) Filed: Dec. 13, 2001

Related U.S. Application Data
(62) Division of application No. 09/464,285, filed on Dec. 15, 1999.

(51) Int. Cl. A61M 25/01; A61M 25/02; A61M 25/04; A61M 25/06; A61M 25/08; A61M 25/082; A61M 25/085; A61M 25/088; A61M 25/09; A61M 25/095; A61M 25/098

(52) U.S. Cl. 604/528

(57) ABSTRACT

An improved catheter assembly and method are provided for delivering guide wires to vessels. The catheter assembly of the present invention has the feature of containing two guide wire lumens in a single catheter to prevent wire wrapping and crossing of the wires. A torquing member is provided to assist in rotation of the catheter. The assembly allows for the delivery of a wire into a side branch vessel that is at a steep angle with respect to a main vessel. The assembly also provides for the delivery of two guide wires to a bifurcation.

### BACKGROUND OF THE INVENTION

The present invention relates to a catheter assembly and method of use. More particularly, the present invention relates to a catheter assembly and method for assisting in the delivery of a wire into an acutely angulated side branch vessel for the purpose of facilitating further interventional treatment, such as a percutaneous transluminal coronary angioplasty (PTCA) procedure.

In a PTCA procedure, a balloon catheter is used to dilate the lumen of a coronary artery which has become narrowed or restricted due to the accumulation of atherosclerotic plaque along the artery wall. A balloon catheter is advanced through the vasculature to the stenosis and the balloon is inflated to radially compress the atherosclerotic plaque against the inside of the artery wall. The balloon is then deflated so that the dilation catheter can be removed and blood flow resumed through the dilated artery.

Occasionally, the inflation of the balloon within the artery lumen will dissect either the stenotic plaque or the intima of the blood vessel or both. After the balloon is deflated and removed, blood can flow between the arterial wall and the dissected lining thereby constricting the flow passage or causing a section of the dissected lining, commonly called an “intimal flap,” to be forced into the flow passageway. In the event of partial or total occlusion of a coronary artery by a dissected arterial lining, the patient is put in an extremely dangerous situation requiring immediate medical attention, particularly when the occlusion occurs in one of the coronary arteries.

Another problem which frequently arises after an angioplasty procedure is the appearance of a restenosis at or near the site of the treated artery. The restenosis may appear due to the accumulation of additional atherosclerotic plaque or may be the result of weakened arterial walls which have collapsed inward to restrict blood flow. When restenosis appears, the treated patient may require an additional angioplasty procedure or other treatment such as by-pass surgery, if an additional angioplasty procedure is not warranted.

Due to the problems caused by dissections of the arterial lining or the appearance of restenosis, much research has been performed on ways to maintain the patency of an artery after the angioplasty procedure is completed. In recent years, expandable endoprosthetic devices, commonly called “stents,” have gained widespread acceptance as a means to support the arterial walls and maintain the patency of a treated vessel. Stents are generally cylindrically shaped intravascular devices which are placed within a damaged artery to hold it open and maintain unimpeded blood flow. Stents prevent dissected arterial linings from occluding an artery by pressing the dissected tissue against the arterial wall until natural healing results in the re-securing of the dissected tissue to the arterial wall. Stents also prevent the appearance of restenosis in the treated vessel by supporting the weakened arterial walls.

Various means have been developed for delivering and implanting intravascular stents within a body lumen. One common method involves compressing or otherwise reducing the diameter of a self-expanding stent, mounting the compressed stent on the distal end of a delivery catheter, placing a tubular sheath over the stent to restrain the stent in the contracted condition, and advancing the catheter through the patient's vasculature to the desired location. Once the stent is properly positioned, the stent is exposed by withdrawing the sheath proximally with respect to the stent, advancing the stent distally with respect to the sheath, or performing a combination of both. Once free from the sheath, the self-expanding stent expands against the arterial walls to thereby hold open the artery or other body lumen into which it is placed.

One of the difficulties with some interventional procedures, such as stenting procedures, involves accessing an acutely angulated side branch vessel with a wire. These acutely angulated side branch vessels are often not accessible due to prolapse of the wire as it attempts to make a steep bend and enter the side branch from the main vessel.

Additionally, it is often desirable to place two wires from outside the body to a position beyond a bifurcation for the purpose of further interventional treatment. Some prior art concepts require bringing a catheter assembly into the body over two wires. The delivery of such prior devices to the bifurcation is highly unreliable and often unsuccessful because of the wrapping of the second wire with the first at various points between its entry into the body and its arrival at the bifurcation. It is impossible to steer a second wire under fluoroscopy without crossing first in front of, then behind the first wire as the second wire reaches tortuosity. When such a prior art catheter assembly encounters a wrap, it fails to advance and the delivery of such a device is unsuccessful. The present invention solves these and other problems.

As used herein, the terms “proximal,” “proximally,” and “proximal direction” when used with respect to the invention are intended to mean moving away from or out of the patient, and the terms “distal,” “distally,” and “distal direction” when used with respect to the invention are intended to mean moving toward or into the patient. These definitions will apply with reference to apparatus, such as catheters, guide wires, stents, the like. When used with reference to body lumens, such as blood vessels and the like, the terms “proximal,” “proximally,” and “proximal direction” are intended to mean toward the heart; and the terms “distal,” “distally,” and “distal direction” are intended to mean away from the heart, and particularly with respect to a bifurcated blood vessel, are intended to mean in the direction in which the branching occurs.

### SUMMARY OF THE INVENTION

The invention provides for an improved catheter assembly and method of use for assisting in the delivery of a wire into an acutely angulated side branch vessel for the purpose of facilitating further interventional treatment. The catheter assembly of the present invention has the feature of containing, in addition to a tracking guide wire, an integrated positioning guide wire and torquing member that affect rotation and precise positioning of the assembly.

In one aspect of the invention, there is provided a catheter assembly that includes an elongate catheter having a tracking guide wire lumen and a positioning guide wire lumen. A torquing member is associated with the tracking guide wire lumen and positioning guide wire lumen so that as the catheter is positioned in a body lumen, the torquing member assists in properly orienting the catheter in the lumen.

In another aspect of the invention, there is provided a method of preparing a vessel for interventional treatment. The method includes the steps of providing a tracking guide wire and tracking guide wire lumen; providing a positioning guide wire and positioning guide wire lumen; providing a torquing member; torquing the positioning guide wire relative to the tracking guide wire with the assistance of the torquing member; and rotating a catheter into a desired position within the vessel and delivering the positioning wire into a side branch vessel. Withdrawal of the catheter and the tracking wire then allows the positioning wire to remain in the side branch providing side branch access for interventional treatment to that vessel. Moreover, withdrawal of the catheter leaving the tracking and positioning wires in place permits the subsequent use of these two unwrapped wires should further intervention requiring two wires be necessary.

Other features and advantages of the present invention will become apparent from the following detailed description, taken in conjunction with the accompanying drawings, which illustrate, by way of example, the principles of the invention.

### BRIEF DESCRIPTION OF THE DRAWINGS

FIG. 1 is an elevational view of the catheter distal section depicting the two guide wire delivery system.

FIG. 2 is a longitudinal cross-sectional view of the catheter distal section of FIG. 1 depicting aspects of the invention.

FIG. 3 is a transverse cross-sectional view of the catheter distal section depicting the torquing member.

FIG. 4 is a partial elevational view depicting the torquing member in phantom lines.

FIG. 5 is a partial elevational view depicting the exit port for the positioning guide wire.

FIG. 6 is a partial elevational view depicting a slit associated with the exit port shown in FIG. 5.

FIG. 7 is a longitudinal cross-sectional view of the catheter distal section and the torquing member.

FIG. 8 is a longitudinal cross-sectional view of the catheter distal section and the torquing member.

FIG. 9A is an elevational view of a bifurcation in which a prior art attempt is made to deliver a guide wire to a side branch vessel.

FIG. 9B is an elevational view of a bifurcation in which a prior art attempt is made to deliver a guide wire to a side branch vessel.

FIG. 10 is an elevational view of one embodiment of the catheter assembly of the present invention at a target site showing a guide wire being delivered into a side branch vessel.

FIG. 11 is a longitudinal cross-sectional view of an embodiment of the catheter distal section wherein the tracking guide wire lumen is of the rapid-exchange type.

FIG. 12 is an elevational view one embodiment of the catheter assembly of the present invention at a target site showing an alternative method of delivery of a guide wire into a side branch vessel.

FIG. 13 is an elevational view one embodiment of the catheter assembly of the present invention at a target site showing an alternative method of delivery of a guide wire into a side branch vessel, before rotation of the catheter.

FIG. 14 is an elevational view showing the catheter after rotation has been accomplished.

FIG. 15 is an elevational view depicting the catheter after it has been withdrawn to the steeply angulated side branch vessel.

FIG. 16 is an elevational view depicting two guide wires in the vessel in preparation of further intervention.

### DETAILED DESCRIPTION OF THE PREFERRED EMBODIMENTS

As shown in the exemplary drawings wherein like reference numerals indicate like or corresponding elements among the figures, the present invention includes an improved catheter assembly and method of use for assisting in the delivery of a wire into an acutely angulated side branch vessel for the purpose of facilitating further interventional treatment.

Side branches with very steeply angulated origins, most commonly of the circumflex from the left main, but occasionally of the origin of the LAD, diagonal or a marginal, do not permit wire access. In some instances the tip of the wire cannot be directed into the side branch. More commonly the tip of the wire can be directed into the highly angulated side branch but the soft portion of the wire then herniates and progresses forward in the main branch, not allowing the following stiffer portion of the wire to advance into the angulated side branch. Access for intervention of any design within this side branch is then denied.

As mentioned above, some attempts at providing stenting solutions for bifurcations have implemented the use of two guide wires in the body at once for preparation of interventional treatment at a bifurcation wherein one guide wire is fed into a main vessel and another guide wire is fed into a side branch vessel. When the second of these wires is advanced it is impossible to visually or otherwise prevent this wire from passing first anterior to and then posterior to the first wire, particularly within tortuosity within the guide or in the proximal portion of the native vessel system. This results in wrapping of the wires. Such wrapping becomes evident when an attempt is made to bring a device or devices over these two wires to reach the point of bifurcation. When the device or devices encounter these wraps, resistance to advancement of the device occurs, often not permitting its delivery to the bifurcation.

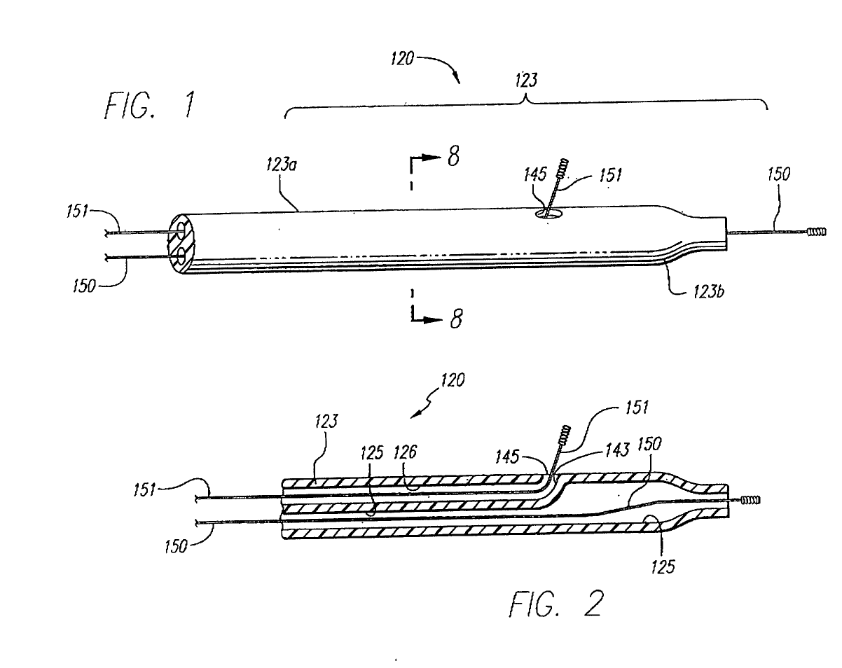

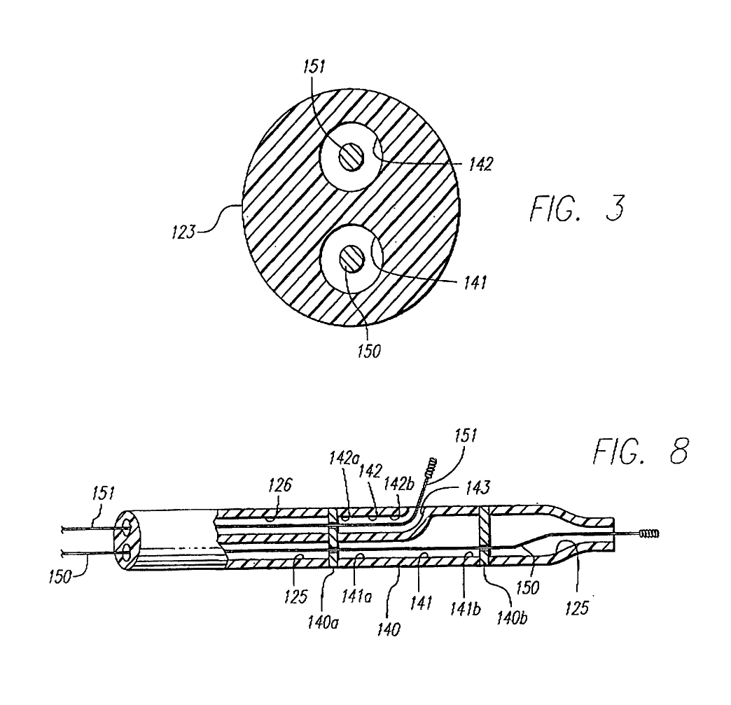

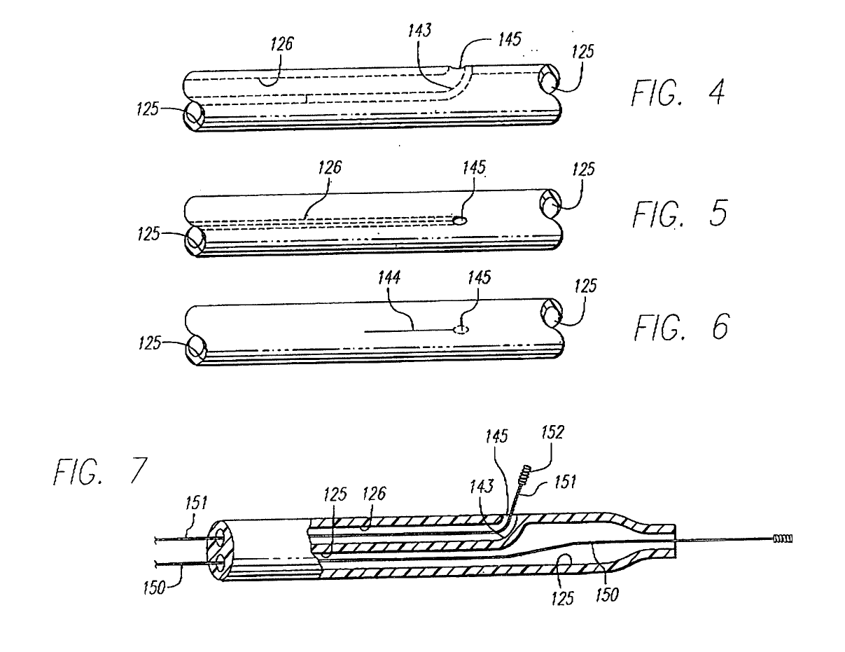

Turning to FIGS. 1-8, in one embodiment of the present invention, a two guide wire catheter assembly 120 is configured to provide maximum torque so that the catheter can be properly positioned in main vessel 6 to deliver integrated positioning guide wire 151 to side branch vessel 5. Referring now to FIGS. 1 and 2, elongate catheter 121 includes a proximal end and distal end 123b. The catheter further is defined by distal section 123 which has a tracking wire lumen 125 and a positioning guide wire lumen 126 extending therethrough.

The present invention provides for a torquing member to assist in torquing the catheter to optimally position the guide wires in the vasculature. A torquing member 140, as shown in FIG. 8, is attached to and aligned with tracking guide wire lumen 125 and positioning guide wire lumen 126. The torquing member can include first port 140a and second port 140b. As depicted in FIG. 8, the torquing member comprises tracking guide wire lumen 141 and positioning guide wire lumen 142. The torquing member positioning guide wire lumen 142 has a proximal end 142a and a distal end 142b while torquing member tracking guide wire lumen 141 has a proximal end 141a and a distal end 141b. The torquing member tracking guide wire lumen proximal end 141a is aligned with the catheter tracking guide wire lumen 125. The torquing member positioning guide wire lumen proximal end 142a is aligned with the catheter positioning guide wire lumen 126. Thus, there is a substantially continuous guide wire lumen for each of the tracking and positioning guide wire lumens that extend through at least a portion of the catheter, through the torquing member, and the tracking guide wire lumen extends distally of the torquing member. The tracking guide wire lumen 125 slidably receives tracking guide wire 150 and positioning guide wire lumen 126 slidably receives positioning guide wire 151. The tracking guide wire slidably extends through the catheter guide wire tracking lumen and through the torquing member guide wire lumen. The positioning guide wire slidably extends through the catheter positioning guide wire lumen and through the torquing member positioning guide wire lumen where it exits into a vessel. The guide wires 150,151 preferably are stiff wires each having a diameter of 0.014 inch, but can have different diameters and degrees of stiffness as required for a particular application. A particularly suitable guide wire can include those manufactured and sold under the tradenames Sport® and Ironman®, manufactured by Advanced Cardiovascular Systems, Incorporated, Santa Clara, Calif.

In keeping with the invention, torquing member 140 further comprises ramp 143 positioned in the positioning guide wire lumen 142. The ramp is positioned in the torquing member and assists the positioning guide wire in advancing through and exiting the catheter. The ramp 143 is sloped and begins a gradual upward slope at the torquing member first port 140a and ends slightly proximal to the torquing member second port 140b. The ramp is distal to the torquing member first port 140a and proximal to the torquing member second port 140b. The ramp ends at opening (or exit port) 145 just proximal to the torquing member second port 140b. The gradual upward slope of the ramp will facilitate the advancement of positioning guide wire 151 so that the guide wire slides up the ramp as it is advanced and it exits the catheter through opening 145 at second port 140b. In this embodiment, the positioning guide wire exits the catheter at a slight angle to a normal to the catheter.

As shown in FIG. 6, the torquing member positioning guide wire lumen 142 preferably has slit 144 in catheter wall 146 located on the side of catheter 121 opposite of opening 145, and is positioned proximal to opening 145. As the positioning guide wire advances through positioning guide wire lumen 126 and slides along ramp 143, it may have a tendency to bend slightly as it encounters frictional resistance along the gradual slope of the ramp. In order to relieve the bending moments in the wire, slit 144 allows the wire to flex into the slit thereby providing a more gradual bend in the positioning guide wire.

The torquing member 140 preferably is formed from a rigid material made from plastic or metal. As shown in FIG. 2, in one embodiment catheter tracking guide wire lumen 125 extends from the torquing member forming a continuous lumen proximal to the torquing member and through it to the catheter distal end.

The catheter distal section 123 extends from proximal end 123a to distal end 123b. The torquing member 140 can be positioned at any point along the catheter distal section.

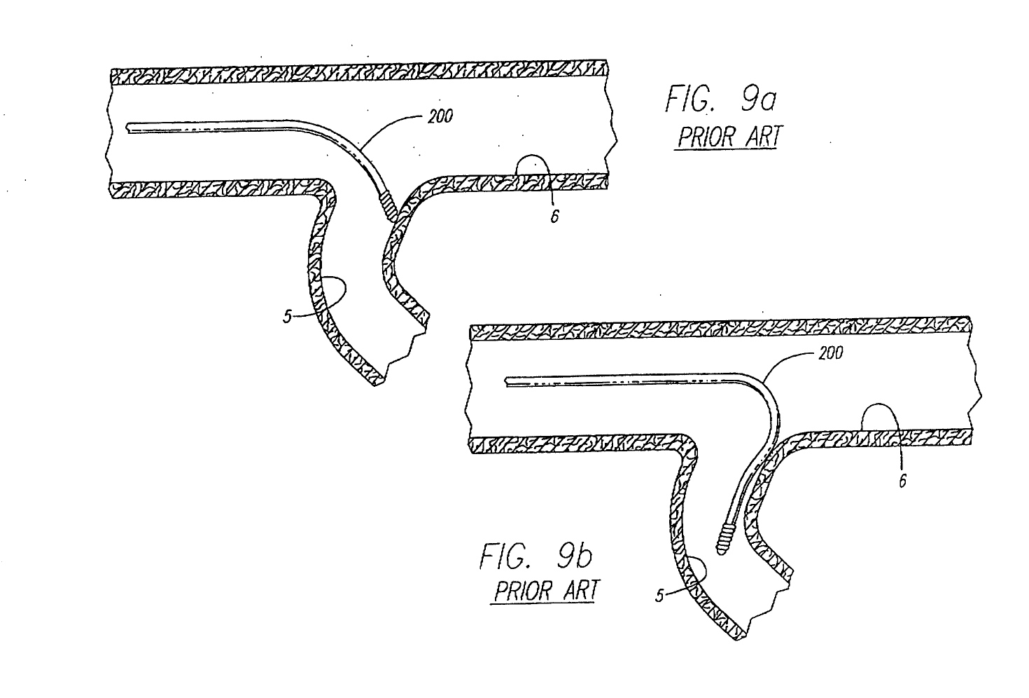

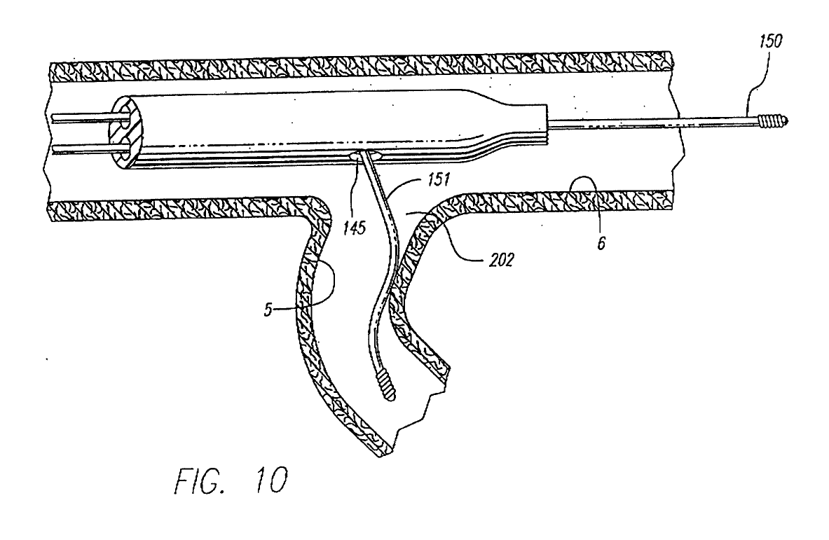

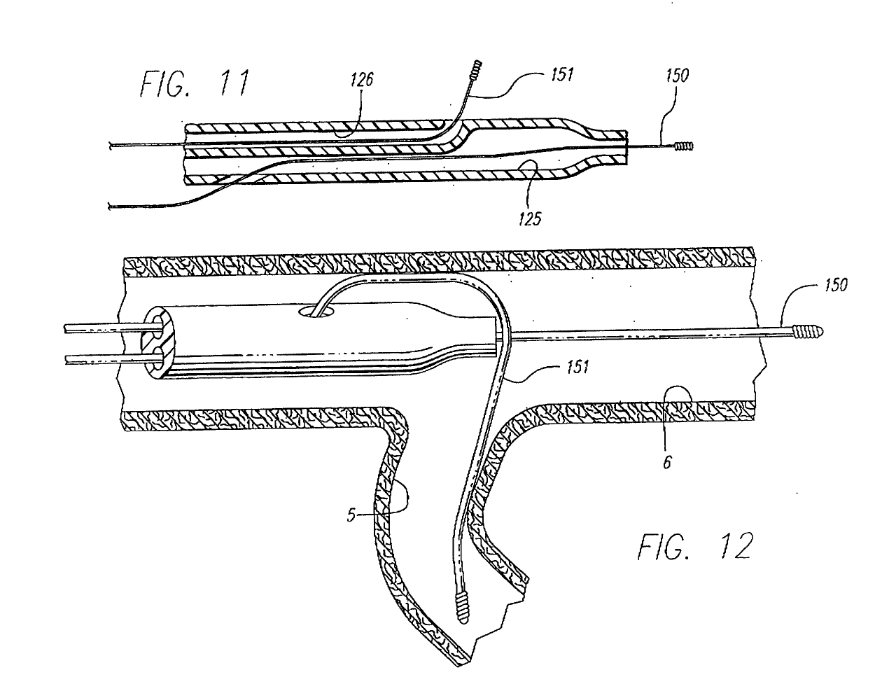

Referring now to FIGS. 9A and 9B, problems seen in the prior art in delivering guide wire 200 from main vessel 6 into side branch vessel 5 can be solved by the present invention. As mentioned above, the guide wire can prolapse and not go into the side branch vessel if the angle of the side branch vessel with respect to the main vessel is too steep.

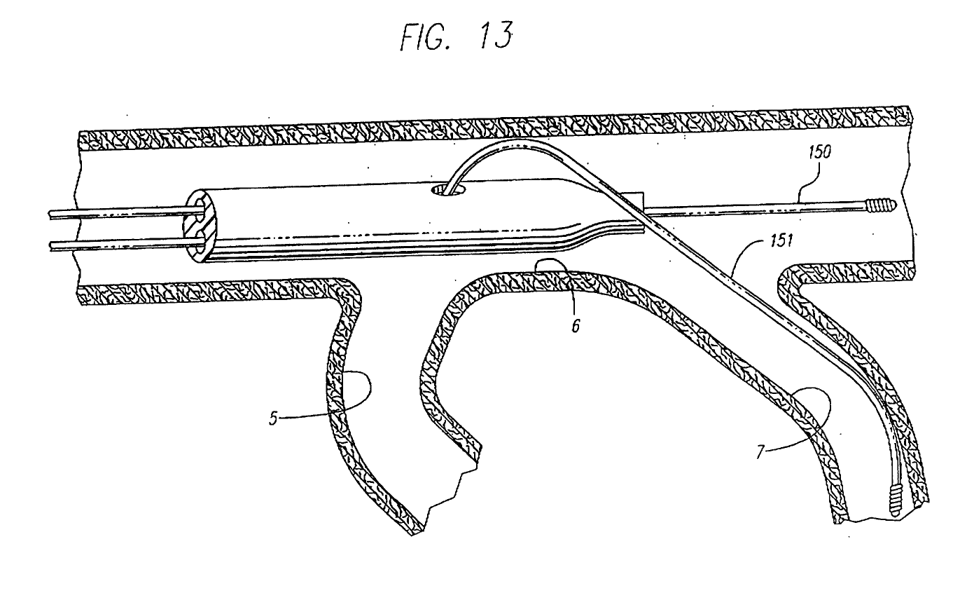

Referring to FIG. 10, in keeping with one method of the invention, the distal end of tracking guide wire 150 is advanced into main vessel 6 and distal to the target area, with the proximal end of the tracking guide wire remaining outside the patient. The distal section 123 of the catheter is then advanced, preferably with the use of a guiding catheter (not shown), along the tracking wire until opening 145 is properly positioned at the target area. Up to this point, positioning guide wire 151 resides in positioning guide wire lumen 126 so that the distal end of the positioning wire preferably is near opening 145. This method of delivery prevents the two guide wires from wrapping around each other, the positioning wire being protected by the catheter assembly during delivery.

The distal end of positioning guide wire 151 is then advanced by having the physician push the proximal end from outside the body. The distal end of the integrated positioning guide wire travels through positioning guide wire lumen 126, up ramp 143 whereby the wire is forced to move radially outwardly, and out of opening 145. Preferably, opening 145 is already somewhat aligned with origin 202 of side branch vessel 5. If not, then some rotation and longitudinal displacement of assembly 120 may be needed in order to advance the positioning guide wire through origin 202 and into the side branch vessel.

After positioning guide wire 151 is advanced into side branch vessel 5, the physician further advances assembly 120 in the distal direction. Due to the assistance of torquing member 140, this action causes the positioning guide wire to push against a wall of the side branch vessel, thus creating a torquing force in the positioning guide wire relative to tracking guide wire 150. This torquing force acts to rotate assembly 120 such that opening 145 comes into alignment with origin 202 of the side branch vessel. The positioning guide wire can then be further advanced into the side branch vessel.

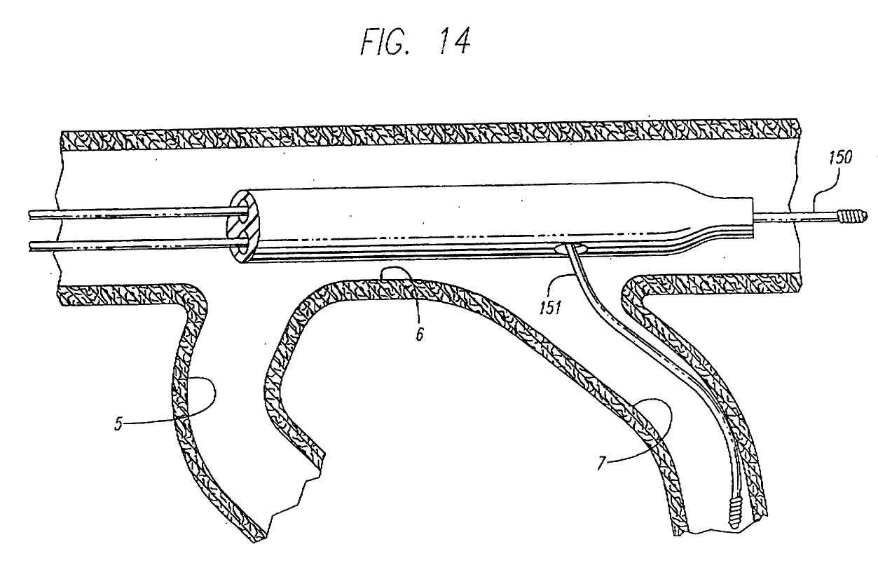

Thereafter, catheter assembly 120 is withdrawn from the patient's vasculature. The tracking guide wire 150 can be withdrawn with the catheter assembly. The positioning guide wire 151 can be left within side branch vessel for further interventional treatment, such as the delivery of a stent. As shown in FIG. 11, in one embodiment tracking guide wire lumen 125 is of the rapid-exchange (RX) type (or unzippable-rapid-exchange type) and positioning guide wire lumen 126 is of the over-the-wire (OTW) type, which are known in the art. However, the catheter assembly can be designed so that one or both of tracking guide wire 150 and positioning guide wire 151 can be unzipped through slits (not shown) from the catheter thereby allowing both wires to act as a rapid exchange wires. It is also contemplated that one or both lumens can be of the over-the-wire type.

Referring to FIG. 12, at times assembly 120 may be delivered to the target site such that such that opening 145 is facing away from side branch vessel 5. In this case, positioning guide wire 151 may be deflected off of the wall of the main vessel and into the side branch vessel. After the positioning guide wire is advanced into the side branch vessel, the physician further advances assembly 120 in the distal direction. As described previously, due to the assistance of torquing member 140, this action causes the positioning guide wire to push against a wall of the side branch vessel, thus creating a torquing force in the positioning guide wire relative to tracking guide wire 150. This torquing force acts to rotate the assembly into the proper position.

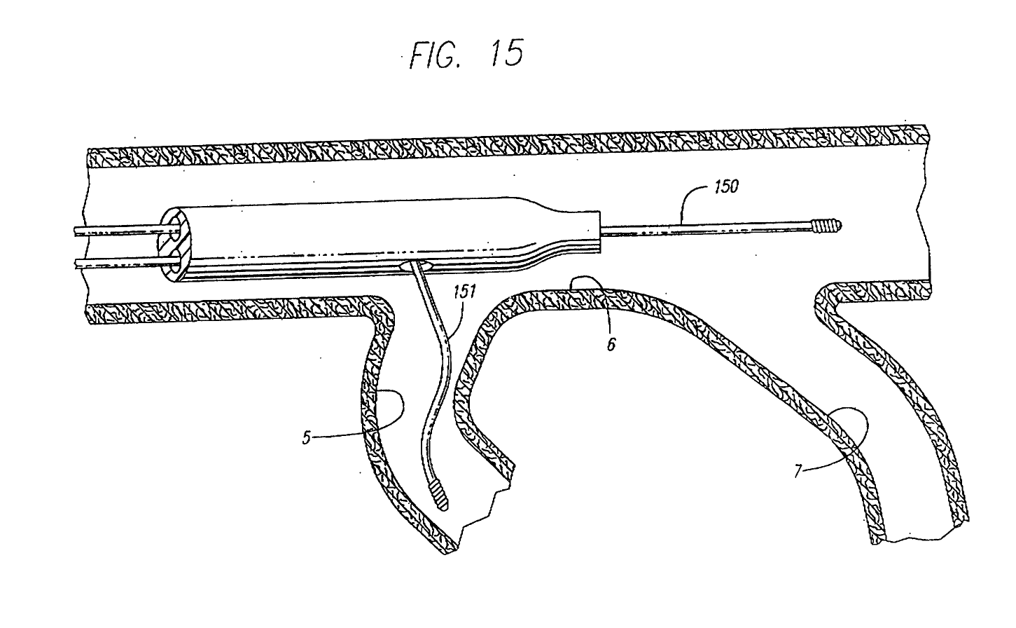

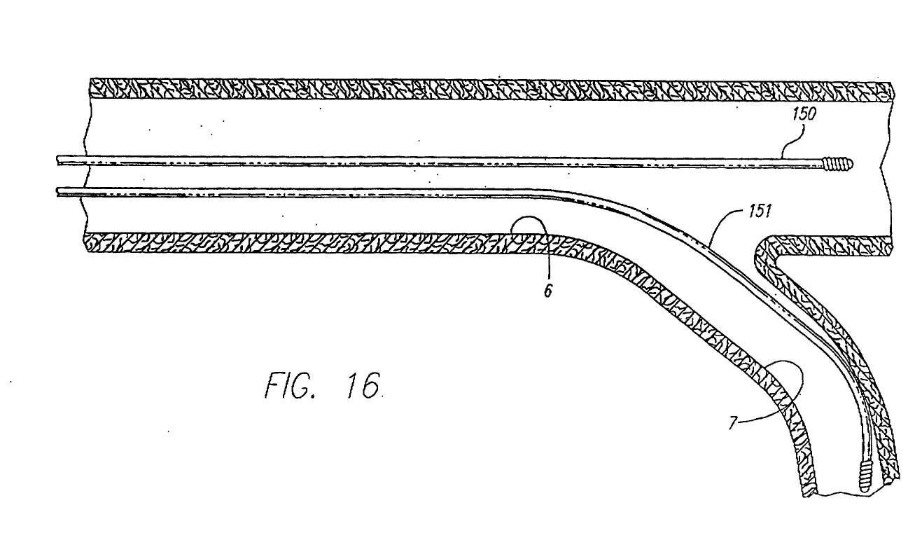

In some instances, however, the unfavorable orientation of opening 145 to face away from side branch vessel 5 may not permit advancement of positioning wire 151. In this circumstance the physician may choose, as shown in FIG. 13, to advance the positioning wire into distal side branch vessel 7 that possesses a shallower angle with respect to the main vessel but has an origin on the same side of the main vessel as the target vessel. The physician may then advance catheter assembly 120 distally causing the assembly to rotate into a position with opening 145 facing side branch vessel 7, with advancement of the catheter arrested at the point that the positioning wire is apposed to this bifurcation as seen in FIG. 14. The catheter assembly is then withdrawn until opening 145 is adjacent to or slightly proximal to target side branch 5. The positioning guide wire 151 is then first withdrawn and then advanced into side branch vessel 5 as shown in FIG. 15.

It should be noted that access of side branch 5 by positioning wire 151 (as shown in FIG. 15) is aided not only by the more favorable angle of entry created by ramp 143, but also by the elimination of prolapse of the soft portion of positioning wire 151 into main vessel 6 accomplished by its exit from the catheter assembly 120, further supported by the stiff aspect of tracking wire 150.

If the bifurcation between main vessel 6 and side branch 7 were targeted for subsequent intervention using one or more stent devices, catheter assembly 120 would be withdrawn after rotation into position as shown in FIG. 14, leaving both the tracking wire and the positioning wire in position as shown in FIG. 16. The delivery of one or more devices over these two unwrapped wires would then be possible without encountering wire wrap.

While the invention herein has been illustrated and described in terms of a catheter assembly and method of use, it will be apparent to those skilled in the art that the invention can be used in other instances. Other modifications and improvements may be made without departing from the scope of the invention.

What is claimed:

1. A catheter assembly, comprising:
an elongate catheter having a tracking guide wire lumen and a positioning guide wire lumen; and
a torquing member associated with the tracking guide wire lumen and positioning guide wire lumen so that as the catheter is positioned in a body lumen, the torquing member assists in properly orienting the catheter in the lumen.

2. The assembly of claim 1, wherein the tracking guide wire lumen slidably receives a tracking guide wire.

3. The assembly of claim 1, wherein the positioning guide wire lumen slidably receives a positioning guide wire.

4. The assembly of claim 1, wherein the torquing member includes a tracking guide wire lumen aligned with the catheter tracking guide wire lumen, and a positioning guide wire lumen aligned with the catheter positioning guide wire lumen.

5. The assembly of claim 4, wherein the torquing member further includes a ramp positioned in the positioning guide wire lumen.

6. The assembly of claim 1, wherein the torquing member is formed from a rigid material.

7. The assembly of claim 6, wherein the torquing member is formed from a plastic material.

8. The assembly of claim 6, wherein the torquing member is formed from metal.

9. The assembly of claim 3, wherein the catheter positioning guide wire lumen includes a slit to permit the positioning guide wire to bow through the slit during a catheter positioning procedure.

10. The assembly of claim 1, wherein the catheter tracking guide wire lumen extends from a proximal end through the torquing member and through a distal end of the catheter.

11. The assembly of claim 10, wherein the catheter tracking guide wire lumen is in fluid communication with the torquing member tracking guide wire lumen and the catheter positioning guide wire lumen is in fluid communication with the torquing member positioning guide wire lumen.

12. The assembly of claim 1, wherein the elongate catheter has a distal section.

13. The assembly of claim 12, wherein the torquing member is positioned at a proximal end of the catheter distal section.

14. The assembly of claim 12, wherein the torquing member is positioned at a distal end of the catheter distal section.

15. A method of preparing a vessel for interventional treatment, comprising the steps of:
providing a tracking guide wire and tracking guide wire lumen;
providing a positioning guide wire and positioning guide wire lumen;
providing a torquing member;
torquing the positioning guide wire relative to the tracking guide wire with the assistance of the torquing member; and
rotating a catheter into a desired position within the vessel and delivering the positioning wire into a side branch vessel.

16. The method of claim 15, further including the step of withdrawing the catheter, thereby leaving the positioning guide wire and the tracking guide wire within the vessel, without wrapping of the positioning guide wire and the tracking guide wire.

17. The method of claim 16, further including the step of withdrawing the tracking guide wire.
